(19) United States
(12) Patent Application Publication
(10) Pub. No.: US 2020/0132665 A1
(43) Pub. Date: Apr. 30, 2020

(54) Method for in vitro detection of the proarrhythmogenic risk of a drug candidate on human induced pluripotent stem cell-derived cardiomyocytes (hiPSC-CM)

(71) Applicant: Universitatea din Bucuresti, Bucuresti (RO)

(72) Inventors: Bogdan AMUZESCU, Bucuresti (RO); Beatrice Mihaela RADU, Bucuresti (RO); Dan Florin MIHAILESCU, Chitila (RO); Stefan Alexander MANN, Frechen (DE)

(21) Appl. No.: 16/598,736

(22) Filed: Oct. 10, 2019

(30) Foreign Application Priority Data
Oct. 24, 2018 (EP) 18465611.4

(51) Int. Cl. G01N 33/487; G01N 33/50; C12N 5/077; G16B 40/10; G06F 17/18; G16B 5/00

(52) U.S. Cl. CPC G01N 33/48728; G01N 33/5061; G01N 33/5032; G01N 2500/10; G16B 40/10; G06F 17/18; G16B 5/00; C12N 5/0657

(57) ABSTRACT

This patent relates to a method of detecting in vitro the proarrhythmogenic risk of a drug candidate performed on human induced pluripotent stem cell-derived cardiomyocytes (hiPSC-CM), where the experimental measurements and recordings of at least five ion currents and of externally paced action potentials (AP) are carried out on a high-performance automated patch-clamp platform before and after application of the candidate drug, and an advanced cardiomyocyte electrophysiology mathematical model is used to accurately reproduce the shape of experimentally recorded externally paced action potential and to generate a restauration stimulus file including the sum of ion currents inhibited by the drug to be applied to the hiPSC-CM in order to restore the initial externally paced action potential, and further used to compute metrics predictive of said proarrhythmogenic risk.

### FIELD OF THE INVENTION

The invention is related to cardiac safety testing, particularly in the field of detecting proarrhythmogenic risk of a drug candidate using an in vitro method on human induced pluripotent stem cell-derived cardiomyocytes (hiPSC-CM).

### BACKGROUND OF THE INVENTION

In the field of cardiac safety testing there is a constant evolution from empirical tests based on experimental evidence to detailed in-depth approaches that offer an abundance of specific quantitative data using in vitro methods.

One of the hot areas of cardiac safety testing is the propensity of medicinal products to induce ventricular cardiac arrhythmia. Older assessment methods were based on prolongation of QT interval, representing the time between the start of the Q wave and the end of the T wave in the heart's electrical cycle as measured through electrocardiography (ECG), thus using in vivo testing of the candidate drugs.

Whereas many experts in the field and big pharma companies consider direct in vivo testing of new drugs on conscious animals via ECG telemetry or on healthy human volunteers as “golden standard”, it can be scientifically proved that effects recorded via methods such as “corrected QT interval prolongation” are often non-specific and devoid of predictive power, let aside the important health-related risks and hazards for healthy human volunteers directly subjected to new pharmacological compounds with insufficiently characterized or completely unknown toxicological profile (1).

Over time, a large number of in vitro preparations have been successfully used to evaluate different proarrhythmogenic effects of candidate drugs, starting with the artificially perfused isolated heart model developed by Oskar Langendorff and its subsequent modern variants [“working heart” ], continuing with “myocardial wedge” preparations, isolated papillary muscle or isolated Purkinje fibers approached by two-microelectrode voltage-clamp, considered the most sensitive to pharmacological effects due high ion channel surface densities required to ensure high velocities of action potential (AP) propagation (2).

Although still highly used in pharmacology assays, these methods and preparations feature a series of disadvantages and inconveniences, such as large amounts of residual tissue acting like reservoirs or “sinks” for drugs, lack of reproducibility and specificity, as well as the need to use large groups of experimental animals that are sacrificed, incurring high prices and low economic effectiveness. There are also differences concerning pharmacological responses and arrhythmogenesis mechanisms between human and animal myocardial tissue, therefore results obtained using animal preparations are provisional and require further confirmation by trials in humans.

For these reasons, a simplified in vitro test was developed, namely screening of the effects of medicinal products on hERG (human ether-à-go-go related gene) delayed rectifier K⁺ channels, which represent the substrate of rapid delayed rectifier K⁺ current (I_{Kr}), the main repolarizing ion current in cardiomyocytes, and further using specific highly sensitive voltage-clamp protocols applied to hERG channels expressed in heterologous systems such as HEK293 cells (human embryo kidney—a diploid cell line). This simplified test gained in popularity and became the method of election for cardiac safety drug testing due to simplicity and ease of automation (3).

This success is a consequence of the fact that most drug-induced arrhythmias consist in torsades-de-pointes (TdP) or polymorphic ventricular tachycardia, resulting in turn from synchronization of early afterdepolarizations (EAD), a frequent proarrhythmogenic condition at single-cardiomyocyte level, and most afterdepolarizations occur as a result of hERG channels block.

Most in vivo and in vitro cardiac safety tests, measuring QT interval prolongation on electrocardiogram or its equivalent in myocardial tissue approached with multielectrode arrays (MEA), belong to the “hERG-centric” paradigm, together with direct hERG inhibition in vitro tests.

The success of the “hERG-centric” cardiac safety testing paradigm has been tremendous: since its adoption in 2005 via ICH (International Conference on Harmonization of Technical Requirements for Registration of Pharmaceuticals for Human Use) standards S7B (for non-clinical in vivo and in vitro tests) and E14 (for clinical trials) not a single case of withdrawal from markets of a drug due to lethal arrhythmia accidents occurred.

However, the “hERG-centric” paradigm is far from being perfect, as revealed recently by the MICE (Multiple Ion Channel Effects) study performed at the drug screening company ChanTest (4). This study demonstrated convincingly that hERG screening alone (or its QT prolongation substitutes) is not 100% predictive for torsadogenic effects. A good counterexample is verapamil, a hERG-blocking compound that is not torsadogenic, because it also blocks L-type Ca²⁺ channels with similar potency, and these two blocking effects of an outward and of an inward ion current balance and compensate each other.

This led to proposal of a new cardiac safety testing paradigm, the CiPA paradigm (Comprehensive in vitro Proarrhythmia Assay), first discussed on Jul. 23, 2013 at a “think tank” meeting organized by FDA (United States Food and Drug Administration) at Silver Spring, Md., and endorsed by a number of drug regulatory agencies and high-profile research institutes (5).

It is known that the presence of a drug candidate may lead to the inhibition of certain ion currents and subsequent distortion of the shape of the externally paced action potential (AP), possibly with appearance of proarrhythmogenic effects such as early or delayed afterdepolarizations.

CiPA proposes an in-depth mechanistic cardiac safety approach based on three steps: 1. study of inhibitory effects of a drug candidate on multiple human cardiac ion currents (corresponding to ion channels) in heterologous expression systems, preferably by automated patch-clamp; 2. use of these inhibition data applied to an advanced human ventricular cardiomyocyte electrophysiology mathematical model, such as the O'Hara-Rudy2011 model (6) to predict proarrhythmogenic events such as early or delayed afterdepolarizations; 3. experimental validation of these predictions via pharmacology experiments on human induced pluripotent stem cell-derived cardiomyocytes (hiPSC-CM) using different methods. The CiPA paradigm may rescue some drug compounds rejected previously based on hERG inhibition criteria, but which are in fact not torsadogenic.

Some progress on the long road of implementing the CiPA paradigm into current practice has been achieved for both experimental and in silico steps (7): new optimized voltage-clamp protocols have been defined (8, 9), Makov gating models for hERG channels have been included in the O'Hara-Rudy model with multiple drug-bound states (10) and temperature dependence (11), and the net charge representing the integral over an entire AP of several inward and outward ion currents has been assessed as a predictor of torsadogenic risk (12).

Several approaches have been proposed for the third step of CiPA, including studies on cardiomyocyte layers approached with multi-electrode arrays (MEA) to measure extracellular local field potentials (13-16) or impedance changes (17-21) or optical recordings on human induced pluripotent stem cell-derived cardiomyocytes hiPSC-CM loaded with voltage-sensitive or calcium-sensitive fluorescent dyes (22-28).

### Disadvantages of Prior Art

CiPA paradigm, representing the closest prior art for the invention (see FIG. 1a), has some disadvantages:

i) Heterologous expression cell lines often express only the main transmembrane subunit of a cardiac ion channel but not the accessory (ancillary) subunits. The accessory subunits exert strong influence on biophysical as well as pharmacological properties, or cellular viability, sealing suitability, or stability in time of the ion current. Thus the results of the assessment of proarrhythmogenic effects of drug candidates may not be satisfactory because of the impossibility to take into consideration all factors that have influence on pharmacological properties.

ii) Although methods known from prior art, such as MEA, are easy to implement and susceptible to automation and high throughput, the information they provide is limited and often equivalent to QT prolongation obtained in in vivo trials, hence devoid of specificity and predictive power. Only global effects on spontaneous pacemaking frequency, overall AP shape and parameters at cardiomyocyte population level, conduction velocity and arrhythmogenesis via reentry mechanisms can be studied by such methods. Detailed characterization of specific inhibitory effects of drug candidates on distinct cardiac ion currents or control of transmembrane potential in measured cells are often impossible.

iii) Other disadvantage of prior art refers to the weaknesses of optical methods used on human induced pluripotent stem cell-derived cardiomyocytes hiPSC-CM loaded with voltage-sensitive or calcium-sensitive fluorescent dyes (22-28) regarding the lack of knowledge of resting potential of the cardiomyocyte (or maximal diastolic potential for pacemaking cells), which is a very important parameter (29), high signal-to-noise ratios, and cell toxicity phenomena upon intense light exposure.

iv) Other optical methods, particularly those developed by Molecular Devices, based on proprietary FLIPR® (fluorescence imaging plate reader) platforms, allow measurement of specific inhibitory effects of drug on K⁺ channels by quantitating Rb⁺ or TI⁺ inflow; although adequate for high-throughput screening, these methods do not achieve an adequate control of transmembrane voltage and are not capable of detailed characterization of effects.

v) Yet other disadvantage of the closest prior art is the length of the detection of the proarrhythmogenic risk, as various steps of the paradigm may take place at different times, not necessarily in the same day.

### Problem Solved by the Invention

The problem to be solved by the invention is how to detect the proarrhythmogenic risk of a drug candidate using a rapid method that is able to process a large quantity of mechanistic data regarding the inhibitory effect of the drug candidate over the cardiomyocytes using a type of cardiomyocyte cells that express not only the main transmembrane subunit of a cardiac ion currents but also the accessory (ancillary) subunits.

### SUMMARY OF THE INVENTION

In order to solve the problem of the invention, the inventors conceived a method of detecting in vitro the proarrhythmogenic risk of a drug candidate where, by use of a high-performance automated patch-clamp platform APCP able to carry out experimental measurements and recordings of at least five human ventricular ion currents and of an externally paced action potential AP performed on human induced pluripotent stem cell-derived cardiomyocytes hiPSC-CM and by use of a computerized algorithm running on a high-performance computer unit HPCU for rapid parameter optimization of an advanced cardiomyocyte electrophysiology mathematical model to accurately reproduce the shape of said externally paced action potential (AP), the following one-shot sequence of steps is carried out on said human induced pluripotent stem cell-derived cardiomyocytes hiPSC-CM:

Step 1—Capturing on a microfluidic chip of one or several human induced pluripotent stem cell-derived cardiomyocytes hiPSC-CM from a suspension, forming of gigaseals, followed by achieving of whole-cell configuration by β-escin perforation, which allows to stabilize the externally paced action potential AP shape over a duration generally between 10 and 20 minutes;

Step 2—Separating, recording, and measuring peaks or steady-state amplitudes of the at least five ion currents by applying to said one or several human induced pluripotent stem cell-derived cardiomyocytes hiPSC-CM approached in β-escin perforated whole-cell patch-clamp of a series of well-defined voltage-clamp protocols;

Step 3—Computing based on automated analysis of recordings using the series of said voltage-clamp protocols of ion conductance surface densities for each of the at least five said ion currents and of scaling factors for ion conductance surface densities of each of said at least five ion currents relative to default values of said advanced cardiomyocyte electrophysiology mathematical model;

Step 4—Measuring and recording an initial externally paced action potential AP of said one or several human induced pluripotent stem cell-derived cardiomyocytes hiPSC-CM by applying a standard current-clamp protocol with a single sweep and with predetermined current stimuli.

Step 5
5a Applying said drug candidate at a given concentration on one or several human induced pluripotent stem cell-derived cardiomyocytes hiPSC-CM fulfilling 2 conditions, namely having a resting potential of of <−50 mV and a having a ventricular-like initial externally paced action potential AP with an Action Potential Duration ratio APD50/APD90>70%, said application of the drug candidate being for a pre-determined period of time, preferably 8 to 15 minutes, and more preferably 10 minutes, and simultaneously, during the same pre-determined period of time:
5b—Measuring and recording the modified externally paced action potential AP under the effect of said drug candidate by applying the same current-clamp protocol as for determining initial externally paced action potential AP, periodically at fixed intervals of time, preferably one-minute, and detecting proarrhythmogenic effects of said candidate drug on the initial externally paced action potential AP shape, such as early or delayed afterdepolarizations;
5c—Rapid optimization of parameters of said advanced cardiomyocyte electrophysiology mathematical model using the said scaling factors for ion conductance surface densities of each of said at least five ion currents in order to accurately reproduce the shape of said externally paced initial action potential AP on human induced pluripotent stem cell-derived cardiomyocytes hiPSC-CM having as result a model-generated shape of said externally paced initial action potential AP;

Step 6—Measuring and recording amplitudes of the at least five ion currents inhibited after application of said drug candidate using the same series of voltage-clamp protocols as in step 2, comparing them with initial amplitudes in the absence of said drug candidate and calculating percentages of inhibition for each of them;

Step 7—Generating a restauration current stimulus file representing the sum of all ion currents inhibited by the candidate drug with reversed sign and the initial predetermined stimuli, to be used further to restore the initial externally paced action potential AP shape in the one or several human induced pluripotent stem cell-derived cardiomyocytes hiPSC-CM subjected to the action of the drug candidate by running a simulation with said advanced cardiomyocyte electrophysiology mathematical model with previously optimized parameters and the percentages of inhibition calculated in the previous step or known in advance from previous experiments;

Step 8—Restoring the modified externally paced action potential AP to its initial shape, recorded before the application of said drug candidate by applying of the restauration current stimulus file using a dynamic-clamp protocol to the same one human induced pluripotent stem cell-derived cardiomyocyte hiPSC-CM on which the drug candidate was applied;

Step 9—Using the calculated percentages of inhibition to compute metrics predictive of proarrhythmogenic risk of said drug candidate such as the net charge integrated over an action potential carried by the sum of several inward and outward ion currents.

### Advantages of the Invention

The main advantages of this invention are the following:

i) The rapidity of the method, as compared with prior art. In less than 30 minutes the detection of the proarrhythmogenic risk of the drug candidate is carried out;

ii) The use of human induced pluripotent stem cell-derived cardiomyocytes hiPSC-CM during all steps of the method carried out on a high-performance automated patch-clamp platform (APCP), as compared with prior art where only some steps of the paradigm are carried out on such cardiomyocytes, allows tests on a cellular preparation expressing not only the main transmembrane subunit of a cardiac ion channel but also the accessory (ancillary) subunits of said cardiac ion channels providing ion currents used by the method;

iii) A better accuracy of detection due to the optimization of the externally paced action potential (AP) and due to the precision of the calculation of the ion conductance surface densities;

iv) The possibility to validate the method of detection of the proarrhythmogenic risk of the drug candidate;

v) The use of β-escin allows to stabilize the shape of the initial externally paced action potential (AP) and the shape of the modified action potential (AP) after application of the drug candidate, said preventing the run-down phenomenon accompanied by progressive externally paced action potential (AP) shortening.

### BRIEF DESCRIPTION OF THE DRAWINGS

FIG. 1a. Diagram of experimental and computation steps according to CiPA paradigm;

FIG. 1 b. Diagram of the method according to the invention;

FIG. 2. Voltage-clamp protocol for measuring peak voltage-dependent Na⁺ current amplitude (Nav_standard_v2_MC defined in CytoLogic);

FIG. 3. Voltage-clamp protocol for separating L-type Ca²⁺ current I_{CaL} from voltage-dependent Na⁺ current I_{Na} (CaVNaVSeparation_MC defined in CytoLogic);

FIG. 4. Voltage-clamp protocol for measuring transient outward current Iₜₒ, delayed rectifier K⁺ current I_{Kr}+I_{Ks} (mainly I_{Kr}), and “funny” hyperpolarization-activated current I_{f} (hERG_Standard_m120_m50_MC defined in CytoLogic);

FIG. 5. (Table 1) Changes and settings applied to the original O'Hara-Rudy2011 C++ script in order to use it for rapid model parameter optimization relative to experimental data during performance of the experimental steps of the method on the automated patch-clamp platform (APCP);

FIG. 6. AP shape obtained by running the modified O'Hara-Rudy2011 model by numeric integration with default parameters for subendocardial, subepicardial, and midmyocardial (M type) human ventricular cardiomyocytes, at physiological temperature (PT=37° C.=310 K)(default temperature in original O'Hara-Rudy2011 model) vs. room temperature (RT=25° C.=298 K);

FIG. 7. Effects on AP shape of varying the scaling factors of several ion conductance densities from 0.3 to 3 times the default values for M type in the O'Hara-Rudy 2011 model;

FIG. 8. (Table 2) Based on analysis shown in previous figure, classification of ion currents and corresponding ion conductance surface densities into components with marked effects on the shape of AP and duration vs. components with reduced effects;

FIG. 9. Results of a computerized parameter optimization algorithm for the modified O'Hara-Rudy 2011 model to fit experimental data, with optimization of different parameters applied in sequential order and computations run on a single processor;

FIG. 10. Flowchart of a parameter optimization algorithm using several rounds of massively parallel computations;

FIG. 11. Results of parameter optimization of the modified O'Hara-Rudy 2011 model using a sequential monoprocessor algorithm to fit experimental data obtained in automated patch-clamp experiments on Cor.4U® cardiomyocytes;

FIG. 12. (Table 3) Comparison of decay rates (in % change/min) between classical “ruptured” whole-cell patch-clamp and β-escin-perforated patch-clamp applied in automated patch-clamp experiments with the CytoPatch™2 platform on Cor.4U® hiPSC-CM, using a complex assay that combines multiple voltage-clamp and current-clamp protocols;

FIG. 13. Comparison of AP plateau run-down and triangulation over 10 min. with current-clamp recordings using a standard protocol: 3 current stimuli of 0.5 ms 2 nA at 3-s interval, first stimulus at 500 ms from start, applied at 1-min, intervals (overlapped traces) in a Cor.4U® cardiomyocyte perforated by classical “ruptured” whole-cell automated patch-clamp (upper panel) with situation when the cardiomyocyte is approached via β-escin-perforated patch-clamp (lower panel).

FIG. 14. A complete run of the method on a Cor.4U® cardiomyocyte approached by automated patch-clamp with a CytoPatch™2 platform in the β-escin-perforated configuration, including AP recordings in initial conditions, after nifedipine application, and during the dynamic-clamp protocol using the restauration current stimulus file.

### LIST OF ABBREVIATIONS (IN ALPHABETIC ORDER)

AP—Externally-paced Action Potential
APCP—Automated patch-clamp platform
APD—[Externally-paced] Action Potential Duration
APD50—[Externally-paced] Action Potential Duration corresponding to 50% repolarization of the membrane of the cardiomyocyte
APD90—[Externally-paced] Action Potential Duration corresponding to 90% repolarization of the membrane of the cardiomyocyte
CiPA—Comprehensive in vitro Proarrhythmia Assay
DAD—Delayed afterdepolarizations
DF—Driving force
EAD—Early afterdepolarizations
ECG—Electrocardiography
hiPSC-CM—Human induced pluripotent stem cell-derived cardiomyocytes
HPCU—High-performance computer unit
I_{CaL}—L-type Ca2+ current
I_{f}— Hyperpolarization-activated (funny) current
I_{K1}— Weak inward rectifier K+ current
I_{Kr}— Rapid delayed rectifier K+ current
I_{Kr}+I_{Ks}—Delayed rectifier K+ current
I_{Ks}— Slow delayed rectifier K+ current
I_{Na}— Voltage-dependent Na+ current
Iₜₒ— Transient outward current
MEA—Multielectrode array
MICE—Multiple Ion Channel Effects
Cₘ— Membrane capacitance
Pₐ— Activation probability
Pₛₛᵢ— Steady-state inactivation probability
PT—Physiological temperature
QT—Time interval between the start of the Q wave and the end of the T wave on electrocardiogram
Rₐ— Access resistance
Rₘ— Membrane resistance
RP—Resting Potential
RT—Room temperature
scgcal—Scaling factor for conductance density for the L-type Ca²⁺ current
scgf—Scaling factor for conductance density for the hyperpolarization-activated (funny) current
scgkr—Scaling factor for conductance density for the rapid delayed rectifier K⁺ current
scgna—Scaling factor for conductance density for the Na⁺ current
scgto—Scaling factor for conductance density for the transient outward current
TdP—Torsades-de-Pointes, the most frequent drug-induced arrhtyhmia
Voff—Voltage offset

### DETAILED DESCRIPTION

The method according to the invention uses two types of equipment. The first is the high-performance automated patch-clamp platform APCP capable of performing usual types of protocols i.e. voltage-clamp protocols, current-clamp protocols, dynamic-clamp protocols, used in the invention to carry out experimental measurements and recordings. The second equipment is the high-performance computer unit HPCU for rapid parameter optimization of an advanced cardiomyocyte electrophysiology mathematical model using an algorithm running on said high-performance computer unit HPCU.

According to the invention, the automated patch-clamp-platform APCP measures and records values of at least five human cardiac ion currents, in short ion currents, and of the externally paced action potential AP. The selection of the ion currents may depend on various criteria, such as but not limited to their relevance to the particular drug candidate and the possibility to measure accurately the values thereof.

The method is conceived to take place in a one-shot sequence of steps, unlike the prior art methods where some steps of the known methods can take place with pauses between them.

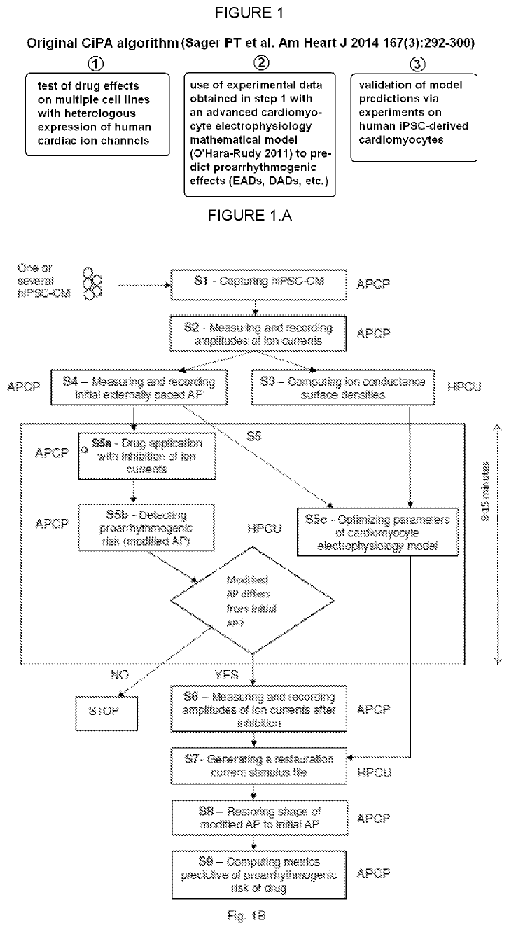

The general flowchart of the method is illustrated in FIG. 1b, in contrast with the general flowchart of the CiPA paradigm from the state-of-art, depicted in FIG. 1a.

The method starts with the preparation steps for the microfluidic chip used by the automated patch-clamp platform: filling with external and internal solutions, priming, voltage offset adjustments and capacitive transient cancellations, measurement of pipette tip resistance on each channel.

Then, in the first step of the method carried out on the automated patch-clamp-platform APCP, one or several human induced pluripotent stem cell-derived cardiomyocytes hiPSC-CM are captured on the microfluidic chip from a suspension, and gigaseals are formed, followed by achievement of whole-cell configuration by β-escin perforation. It is preferable to achieve whole-cell configuration by β-escin perforation in order to stabilize the shape of the externally paced action potential (AP) over a duration generally between 10 and 20 minutes.

The general parameters of the equivalent electrical circuit (membrane capacitance—Cₘ, access resistance—Rₐ, membrane resistance—Rₘ, and voltage offset—V_{off} in current-clamp mode) are monitored; fast and slow capacitive transients are cancelled and series resistance compensation can be applied.

For the β-escin perforated variant a good way to monitor perforation is to apply repeatedly a voltage-clamp protocol with a depolarizing step to elicit fast voltage-dependent Na⁺ current I_{Na}, and to plot its peak amplitude at different moments of time.

Best results in term of seal quality and stability are obtained with a third generation automated patch-clamp platform, e.g. CytoPatch™2, which applies the proprietary Cytocentering® method (patent US20030153067A1) (30) based on small microfluidic chips containing quartz pipette tips with well-defined geometry, with inner tip diameters of 2 or 2.5 μm, surrounded by cytocentering orifices.

The inferior pole of a cell applied in suspension into the transport channel is aspired by the cytocentering orifice and placed tightly over the quartz pipette tip, enabling high quality and stability of gigaseals, using physiological external and internal solutions.

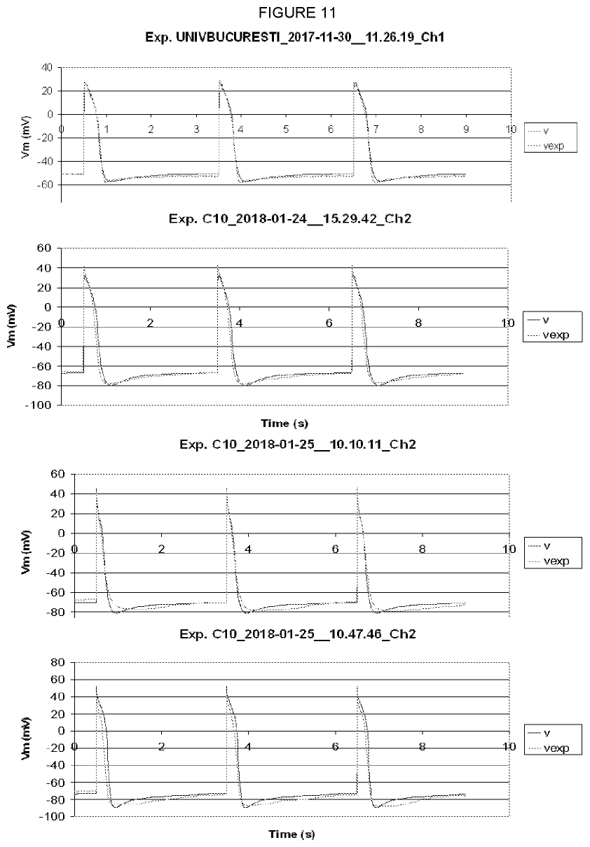

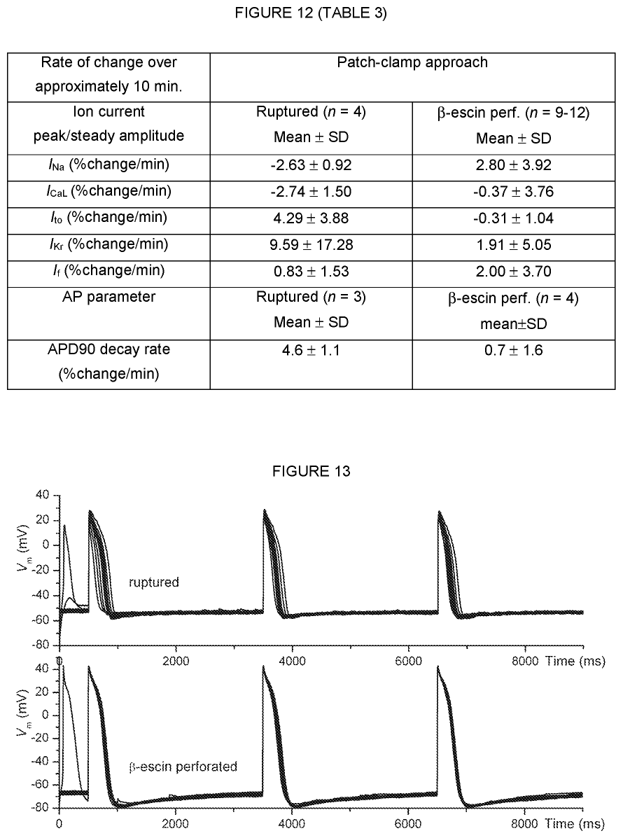

In one preferred embodiment β-escin perforated whole-cell patch-clamp on the one or several human induced pluripotent stem cell-derived cardiomyocytes hiPSC-CM is performed in order to stabilize initial externally paced action potential AP shape and initial peak or steady-state amplitudes of said at least five ion currents over a time interval of 10-20 min. in control conditions, preventing the run-down phenomenon accompanied by progressive shortening of said initial externally paced action potential AP shape that occurs in the classical “ruptured” whole-cell patch-clamp approach on said one or several human induced pluripotent stem cell-derived cardiomyocytes hiPSC-CM (FIG. 12(Table 3) and FIG. 13).

Subsequently, in step 2 of the method, also carried out on the automated patch-clamp-platform APCP, after achieving the whole-cell configuration, a series of voltage-clamp protocols is applied to said one or several human induced pluripotent stem cell-derived cardiomyocytes hiPSC-CM in order to separate, record and measure steady-state amplitudes of the at least five ion currents, and said peak or steady-state amplitudes are measured and the results recorded.

In another preferred embodiment, used hereafter as a non-limiting example of the method, the following human ventricular ion currents are used:
peak rapid voltage-dependent inward Na⁺ current (I_{Na});
peak L-type inward Ca²⁺ current (I_{CaL});
transient outward K⁺ current (Iₜₒ);
“funny” hyperpolarization-activated current (I_{f});
a sum of the rapid (I_{Kr}) and slow (I_{Ks}) components of the delayed rectifier K⁺ current, but where usually I_{Kr} is the dominant component in control conditions, where control conditions mean that no drug candidate was yet applied.

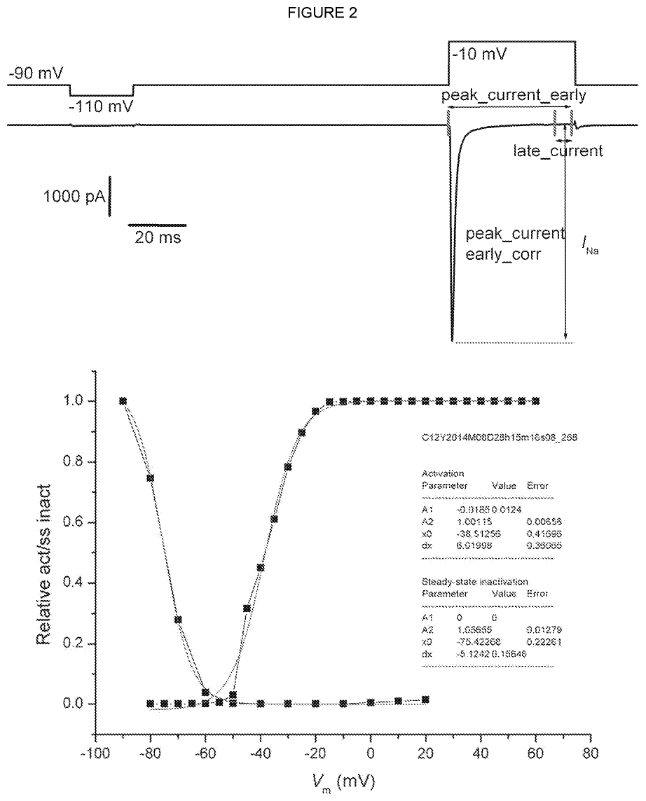

A first voltage-clamp protocol, illustrated in FIG. 2, named Nav_standard_v2_MC as defined in the CytoLogic software that controls the CytoPatch™2 platform, and with the use of which the entire method may be carried out, activates I_{Na} via a depolarizing step at −10 mV from a holding potential of −90 mV.

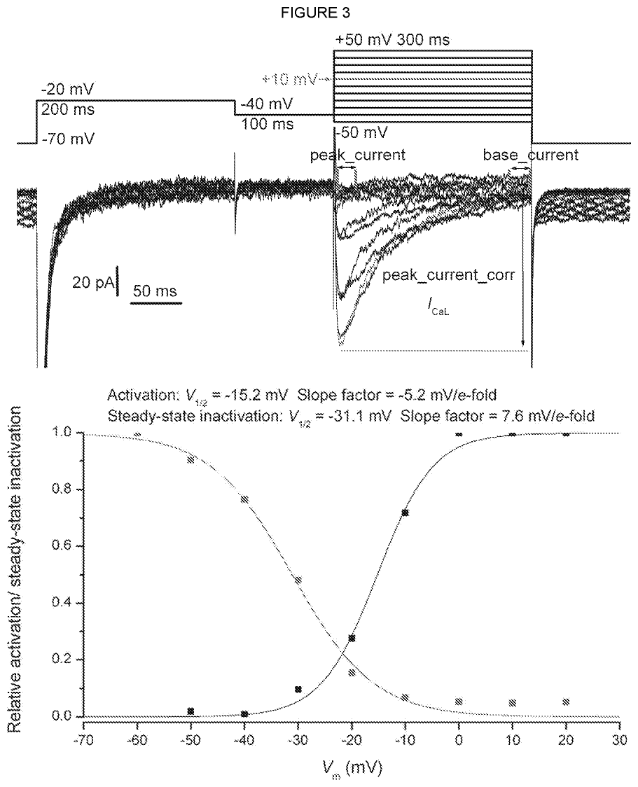

A second voltage-clamp protocol, illustrated in FIG. 3 (CaVNaVSeparation_MC defined in CytoLogic) allows L-type Ca²⁺ ion current I_{CaL} separation from I_{Na} based on different voltage dependencies of steady-state inactivation for these two currents (lower panels of FIG. 2 and FIG. 3).

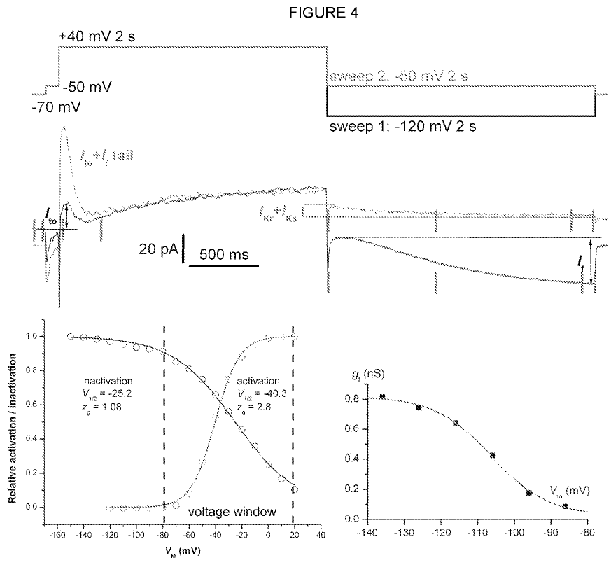

A third voltage-clamp protocol, illustrated in FIG. 4 (hERG_Standard_m120_m50_MC defined in CytoLogic) derived from a standard hERG protocol by adding a supplementary first sweep with the second large voltage step at −120 mV, allows separation and measurement of multiple ion currents: transient outward K⁺ current Iₜₒ at the beginning of the first depolarizing step at +40 mV of the first sweep, the “funny” hyperpolarization-activated current A activated gradually during the 2-s hyperpolarizing step of the first sweep, and the delayed rectifier K⁺ current (a sum of rapid and slow components I_{Kr}+I_{Ks}, but where I_{Kr}, usually predominates and can be approximately estimated) measured as a small peak current at the beginning of the 2-s step at −50 mV of the second sweep, produced by rapid removal from inactivation of delayed rectifier K⁺ channels at this potential, followed by a slow exponential deactivation with similar time constants for I_{Kr} and I_{Ks}.

The above example with three voltage-clamp protocols shall not be considered as limiting, another number of voltage-clamp protocols fall within the scope of the invention as long as the purpose of separating, measuring and recording peak or steady-state amplitudes of the at least five ion currents is achieved.

In step 3 of the method, the recordings previously obtained by initial application of the voltage-clamp protocols are exported and sent to the high-performance computer unit HPCU in an easily readable file format (such as but not limited to Axon Text File—ATF) and automatically analyzed by said high-performance computer unit HPCU with a program, such as for example a C++ script that computes, based on automated analysis of said recordings, ion conductance surface densities for the at least five ion currents, such as for example for I_{Na}, I_{CaL}, Iₜₒ, I_{Kr}, I_{f} using manually entered Cₘ and Rₐ values, by detecting peak current values between well-defined time intervals and base values measured as averages over other well-defined time intervals (limits of intervals used to measure peak and base current values marked with short vertical lines in FIGS. 2, 3, and 4), and applying a series of computations including voltage corrections for liquid junction potential and sometimes for voltage drops across access resistance, computation of driving force (DF) for each ion current component as difference between actual transmembrane voltage and Nernst equilibrium potential (or Goldmann-Hodgkin-Katz potential for I_{f}), obtaining ion conductance surface densities by dividing corrected peak (or steady for I_{f}) current amplitudes to DF and Cm, and supplementary correction by dividing these conductance densities to activation probabilities (Pₐ) and steady-state (removal from) inactivation probabilities (Pₛₛᵢ) for each ion current using Boltzmann charge-voltage functions obtained from own experimental data (as shown in lower panels of FIGS. 2-4) or from literature for Iₜₒ (32).

In addition to ion conductance surface densities, the same program, such as said C++ script computes scaling factors for said ion conductance surface densities for the at least five measured ion currents, such as for example for I_{Na}, I_{CaL}, Iₜₒ, I_{Kr}, I_{f}, relative to default values of said ion conductance surface densities of the advanced cardiomyocyte electrophysiology mathematical model.

In the same afore-mentioned embodiment of the invention the advanced cardiomyocyte electrophysiology mathematical model is O'Hara-Rudy 2011 human ventricular cardiomyocyte model and the scaling factors corresponding to the ion currents selected I_{Na}, I_{CaL}, Iₜₒ, I_{Kr}, I_{f} are named scgna, scgcal, scgto, scgkr, and scgf, respectively. After completing the initial voltage-clamp protocols, the automated patch-clamp platform (APCP) switches to current-clamp mode for carrying out step 4 of the method, in which a standard current-clamp protocol is applied and the initially externally paced action potential (AP) is measured and recorded. Said standard current-clamp protocol is comprised of a single sweep lasting 9 s, and 3 brief current stimuli of 0.5 ms duration and 2 nA amplitude applied at 3-s intervals, the first stimulus being applied at 500 ms from start of recording.

If a current-clamped human induced pluripotent stem cell-derived cardiomyocyte (hiPSC-CM) features spontaneous pacemaking, this activity can be suppressed by applying continuous small hyperpolarizing currents (−10 or −20 pA), as shown in a previous own study (33).

If on one channel of the high-performance automated patch-clamp platform there is at least one human induced pluripotent stem cell-derived cardiomyocyte (hiPSC-CM) with good resting potential and nice ventricular-like initial externally paced action potential AP, the method is continued with steps 5-9. Good resting potential is defined by a value less than −50 mV (<−50 mV), whereas nice ventricular-like initial externally paced action potential AP is considered when it has an APD50/APD90 ratio>70%, where APD stands for Action Potential Duration and the numbers 50, respectively 90 refer to the percentages of repolarization of 50% respectively 90% of the membrane of said cardiomyocyte. Thus the method is continued only for human induced pluripotent stem cell-derived cardiomyocytes hiPSC-CM that satisfy both above-mentioned conditions.

In step 5 of the method three different actions are carried out during a pre-determined period of time, preferably for 8 to 15 minutes, and more preferably for 10 minutes. The example of realization has been carried out for a pre-determined period of time of 10 minutes.

In sub-step 5a, on the automated patch-clamp platform APCP, the candidate drug at a certain concentration is applied for the pre-determined period of time, preferably for 8 to 15 minutes, and more preferably for 10 minutes. If the candidate drug at given concentration blocks or inhibits one or several ion currents, this will distort the shape of the initial externally paced action potential AP, possibly producing proarrhythmogenic effects such as early or delayed afterdepolarizations (EAD or DAD), which per se are an important indication of arrhythmogenic (torsadogenic) risk of the candidate drug.

In sub-step 5b measuring and recording of the modified values of the externally paced action potential AP is carried out on the automated patch-clamp platform APCP in current-clamp mode with the same series of protocols as used for measuring and recording initial externally paced action potential AP, said measurement and recording of the modified values occurring at fixed intervals of time, preferably 1-min.

Thus the detection of the proarrhythmogenic effects of the candidate drug is basically made in step 5b of the method and confirmed in the remainder of the steps.

If the candidate drug has no proarrhythmogenic effects, the measurement and recording carried out in sub-step 5b returns values of the externally paced action potential AP identical to the initial ones as measured and recorded in step 2, in which case the method is stopped. During the pre-determined period of time, preferably for 8 to 15 minutes, and more preferably for 10 minutes, while the candidate drug is applied to the one or several human induced pluripotent stem cell-derived cardiomyocytes hiPSC-CM and pharmacological effects of blocking or inhibition occur, the computerized algorithm running on the high-performance computer unit HPCU is applied in sub-step 5c for rapid optimization of parameters of the advanced cardiomyocyte electrophysiology mathematical model, such as modified O'Hara-Rudy 2011 model, to match the experimentally recorded series of the externally paced action potential AP with the standard stimulation protocol with the model-generated series of externally paced action potentials AP using the same stimulation protocol as in the experimental recording.

In step 6 of the method, the inhibitory effects of the candidate drug on each of the at least five ion currents are measured and recorded by the high-performance automated clamp platform APCP using the same series of voltage-clamp protocols as used in step 2, and the results of the measured and recorded peak or steady-state amplitudes are compared with the values of the initial corresponding amplitudes measured and recorded in the absence of said drug candidate. This step of the method ends with calculation of percentages of inhibition for each of the at least five ion currents.

The calculated percentages of inhibition may be compared, for validation purposes, with known values of percentages of inhibition of each of the at least five ion currents as resulted from other previous experiments that are different from present invention, such as for example experiments carried out on human cardiac ion channels expressed in heterologous systems.

Using either the calculated percentages of inhibition of the previous steps, or the known values of the percentages from other previous experiments, possibly also more complex state-dependent binding data or voltage shifts of activation or steady-state inactivation with more complex mathematical models including Markov models for different ion current components, e.g. a Markov model of hERG published in (10), in step 7 of the method, a restauration stimulus file is generated by the program running on the high-performance computer unit HPCU, preferably the C++ script, in predefined format readable by the software controlling the automated patch-clamp platform APCP by summation of the ion currents generated by the optimized model (read from an output file) with the estimated percentages of inhibition, with reversed sign, because stimulus current by convention has opposite sign to transmembrane current, and said restauration stimulus is added to the initial set of current stimuli of the standard current-clamp protocol (in the example the 3 current stimuli of 0.5 ms 2 nA amplitude at 3-s intervals, first stimulus at 500 ms).

This restauration stimulus file is applied in step 8 of the method to the same one or several human induced pluripotent stem cell-derived cardiomyocytes hiPSC-CM in a dynamic-clamp protocol on the automated patch-clamp platform APCP. The application of said restauration stimulus file has as effect restauration to its initial shape of the modified externally paced action potential AP shape distorted by the inhibition provoked by the candidate drug, by electronically supplying the ion currents that were suppressed by said candidate drug.

In the last step of the method, the 9^{th}, the calculated percentages of inhibition of the previous steps, or the known values of the percentages from other previous experiments as used for the generation of the restauration stimulus file are used to compute on the high-performance computer unit HPCU used in the invention or on another similar equipment metrics predictive of proarrhythmogenic risk of said drug candidate such as the net charge integrated over an action potential carried by the sum of several inward and outward ion currents (12).

According to another embodiment of the invention the optimization of one or several parameters of said advanced cardiomyocyte electrophysiology mathematical model such as but not limiting to scaling factors of conductance surface densities, temperature, is performed in a sequential, parallel, or combined manner on the high-performance computer unit HPCU, taking as optimality criterion the sum of squared differences between the experimentally recorded initial externally paced action potential AP and the model-generated shape of said externally paced initial action potential AP and using some or all the scaling factors previously computed.

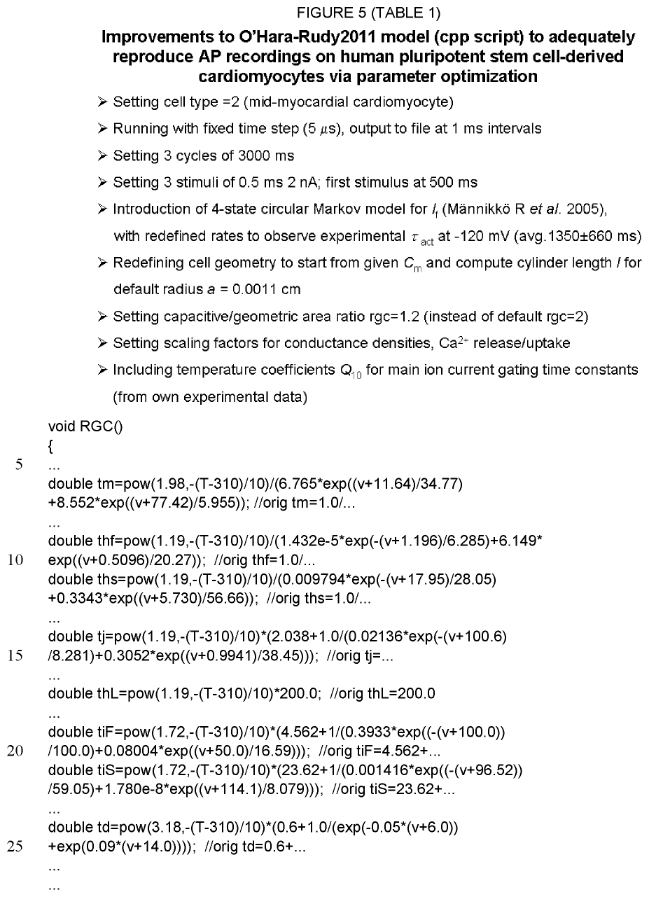

The changes applied to the standard O'Hara-Rudy 2011 model are illustrated in Table 1 (FIG. 5): the most important ones are inclusion of a four-state circular allosteric Markov model of I_{f} gating adapted from (32) with rates adjusted to observe a time constant of I_{f} activation at −120 mV of 1350 ms (average in own experiments on human induced pluripotent stem cell-derived cardiomyocytes (hiPSC-CM) at room temperature) and introduction of temperature dependence of gating for most ion current components using Q₁₀ values obtained from own experiments or from the literature.

Another change applied to the model is use of fixed internal Na⁺ and K⁺ concentrations, equal to concentrations in the internal (pipette) solution, assuming these two cation species diffuse fast enough from pipette to the interior of the cardiomyocyte.

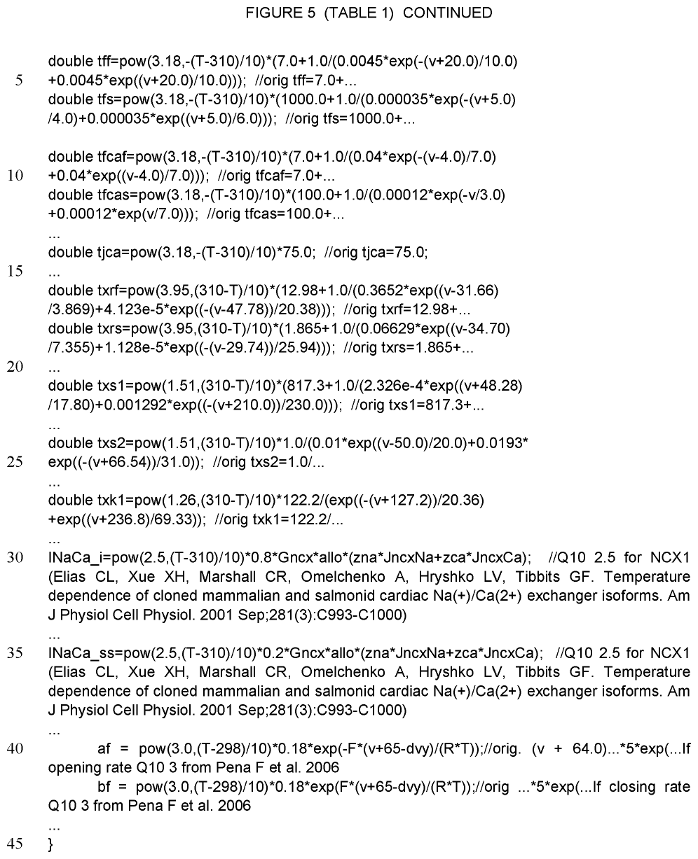

The modified O'Hara-Rudy 2011 model was tested by running numeric integration computations with default stimulus and default parameters for subendocardial, subepicardial and mid-myocardial (M) human ventricular cardiomyocytes, at physiological temperature PT=37° C.=310 K (default temperature in original O'Hara-Rudy 2011 model) vs. room temperature (RT=25° C.=298 K), as shown in FIG. 6: at RT there is a slow-down of AP time course.

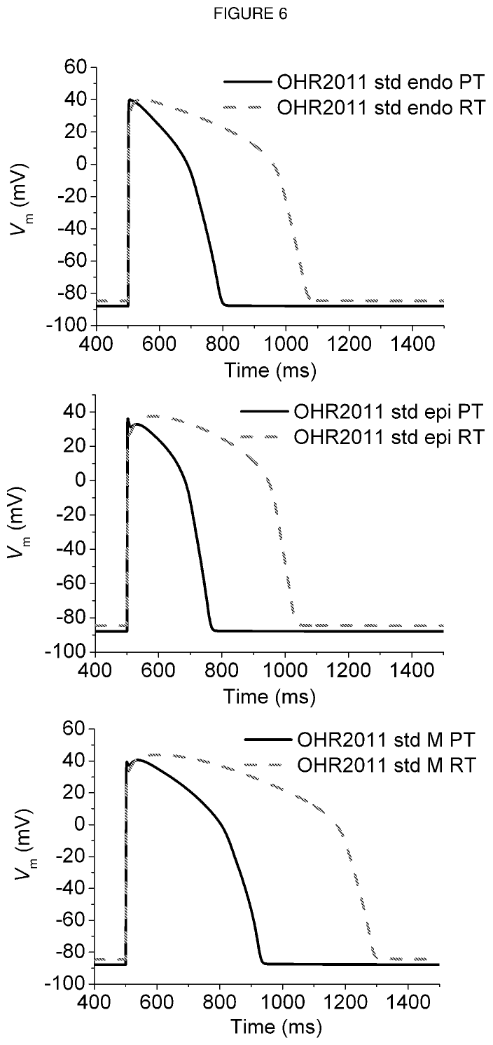

In a set of preliminary tests, the inventors have studied the effects of changing the scaling factors of different ion conductance surface densities on externally paced action potential AP shape and duration; FIG. 7 shows effects on AP, computed using default O'Hara-Rudy 2011 parameter set for M cell type at RT of change between 0.3 to 3 times the default value of conductance density scaling factors scgna, scgcal, scgkr, scgks, scgki, scgf, scgncx, scgnak.

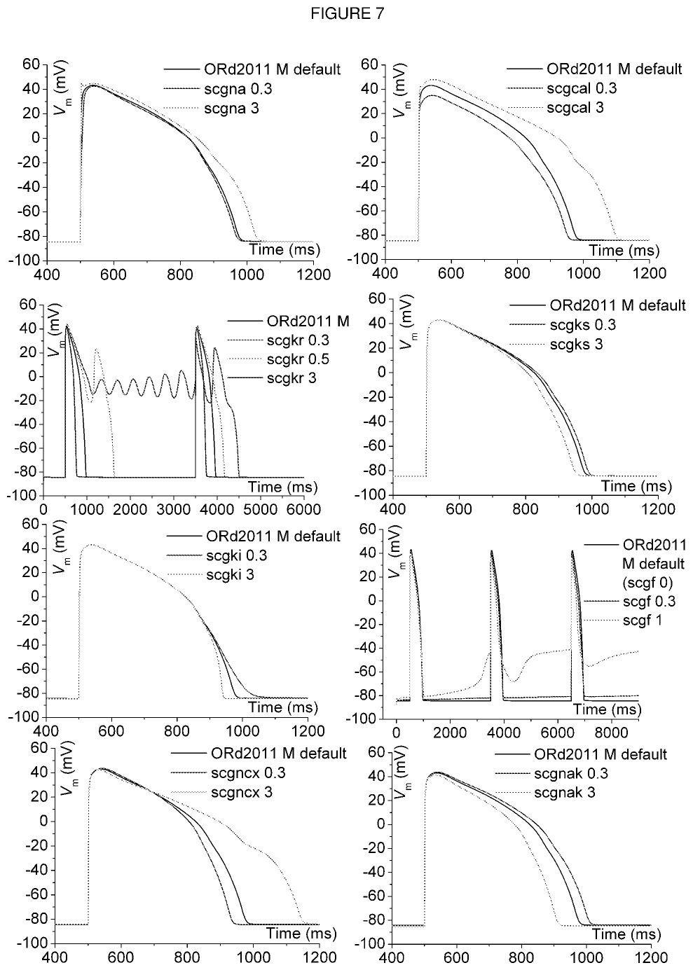

Based of these preliminary tests, in Table 2 (FIG. 8) the different scaling factors in the O'Hara-Rudy model are classified into scaling factors with strong effects on externally paced action potential AP shape and duration vs. scaling factors with reduced effects on AP shape and duration. Based on analysis shown in previous figure, ion currents and corresponding ion conductance surface density scaling factors were classified into components with marked effects on the shape and duration of externally paced action potential AP and components with reduced effects. The rapid optimization algorithms were focused only on components with strong effects on externally paced action potential AP shape.

The most important components with marked effects on the shape and duration of externally paced action potential AP shape may be for example scgkr and scgcal, while scgf exerts strong effects on resting potential.

It is a good approximation for parameter optimization algorithms to vary only the scaling factors with marked effects on externally paced action potential (AP) shape.

There are multiple approaches to computational algorithms for fast parameter optimization of the modified O'Hara-Rudy 2011 model to match experimentally recorded externally paced action potential AP shape in a one or several human induced pluripotent stem cell-derived cardiomyocytes hiPSC-CM.

One approach is to run a sequential optimization algorithm, varying the scaling factors one by one at well-defined intervals and in a well-defined order until getting an optimal value for each of them.

The optimality criterion can be the sum of squared errors, i.e. the sum of squared differences between experimentally recorded transmembrane potential and transmembrane potential generated by the modified O'Hara-Rudy model with a given set of parameters, computed at 1-ms time intervals for a total duration of 9 s, using the same standard current stimulus protocol for both the real one or several human induced pluripotent stem cell-derived cardiomyocytes hiPSC-CM and the model (3 current stimuli of 0.5 ms 2 nA amplitude at 3-s intervals, first stimulus at 500 ms).

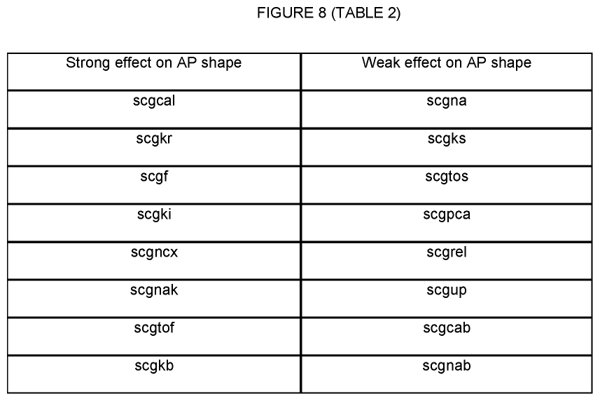

The optimality criterion, such as sum of squared errors for transmembrane potential, landscape for each parameter varied within a certain interval during application of this sequential parameter optimization algorithm for a real experimental dataset is shown in FIG. 9: for most parameters a minimum is attained somewhere within the variation interval. The optimality criterion was the sum of squared errors between experimentally-recorded and model-generated transmembrane potential at 1-ms intervals over a 9-s total interval, with the same stimulation protocol applied in the real experiment and to the model (3 current stimuli of 0.5 ms 2 nA at 3-s interval, first stimulus at 500 ms from start). Each parameter was varied in discrete steps over an interval, and generally there was an optimal value where the sum of squared errors reached a minimum for each of them. The order of parameters within the optimization sequence is extremely important, as are the initial estimates of the other parameters beyond the one being optimized.

Another variant of applying the sequential optimization algorithm is in a parallel manner, using parameter order shuffling schemes to obtain different sequences of parameter optimization on different processors (either main processors or graphical processors).

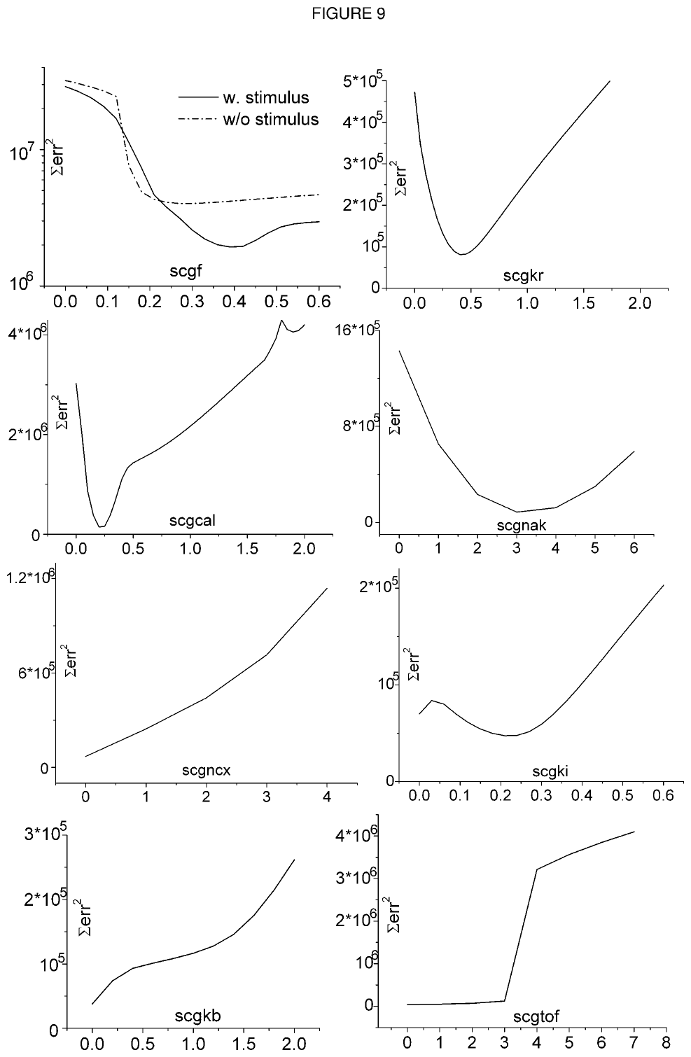

In FIG. 10 the general flowchart of a massively parallel variant of the parameter optimization algorithm is shown, comprised of 5 consecutive rounds run on a majority of the 7168 graphical processors of a system equipped with 2 Nvidia® Tesla® P100 GPU accelerator cards. The algorithm includes 5 rounds of optimization, with well-defined combinations of parameters and intervals of variation for each round, based on preliminary results of optimization algorithms with manual sequential change of parameters. After each round the best-fitting set of parameters is used as base for the next round, although some parameters are finely trimmed during late rounds.

Another effective fast optimization method is to use the initial set of scaling factors computed by the automated analysis script of voltage-clamp recordings in a single round, with adequate guesses for the other scaling factors, to inspect visually the computed externally paced action potential AP shape vs. experimental recorded externally paced action potential AP shape, and to perform manually adjustments of some parameters based on knowledge of effects of each parameter on different AP or resting potential features, followed by a second single round of numeric integration; results obtained with this method, while not perfect, provide usually a satisfactory approximation for generating stimulus files for dynamic-clamp protocols.

Another very computationally-effective variant of the optimization algorithm is to use the experimentally recorded series of externally paced action potential AP shape applied directly to the model with parameters estimated by automated analysis of voltage-clamp recordings or reasonably guessed, and to compute the dynamics of each ion current component during this externally given transmembrane potential, using subsequently the output (dynamics of different ion currents) to generate a stimulus file for dynamic-clamp protocols.

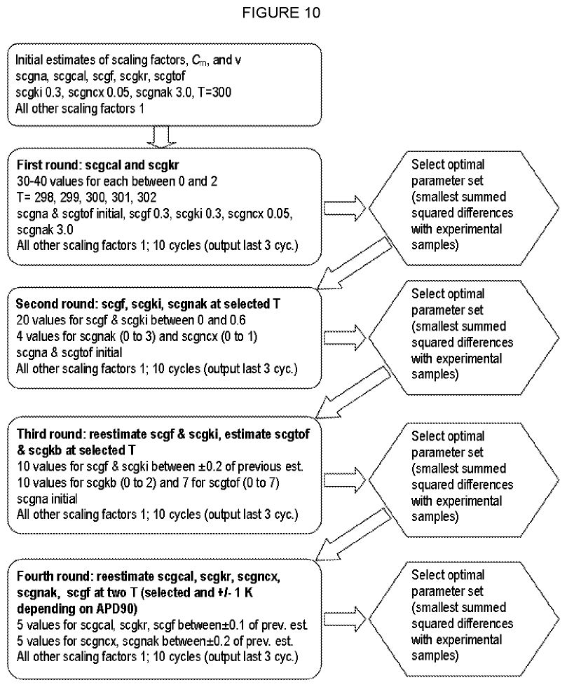

FIG. 11 shows the result of applying the sequential monoprocessor parameter optimization algorithm for 4 distinct workings of the method corresponding to real experiments on one or several human induced pluripotent stem cell-derived cardiomyocytes hiPSC-CM: in each case the externally paced action potential AP shape generated by the optimized model fits satisfactorily the experimentally recorded externally paced action potential AP shape. In all 4 distinct experiments, the experimentally recorded externally paced action potential AP was obtained in current-clamp mode using the standard stimulation protocol (3 current stimuli of 0.5 ms 2 nA at 3-s interval, first stimulus at 500 ms from start).

Table 3 (FIG. 12) shows the advantages of applying the β-escin-perforated method instead of the classical “ruptured” whole-cell patch-clamp approach in experiments on one or several human induced pluripotent stem cell-derived cardiomyocytes hiPSC-CM: the rates of change (in % change/min. relative to initial values) of peak/steady ion current amplitudes are reduced by almost an order of magnitude for the ion current components exerting the most pronounced effects on AP shape and duration: I_{CaL} and I_{Kr}.

A consequence is the improved stability of AP shape and duration in the β-escin-perforated vs. the classical “ruptured” configuration, reflected in the APD90 rate of change (% change/min.) of 0.7%/min. (perforated) vs. 4.6%/min. (“ruptured”) (see Table 3 (FIG. 12)), and as shown in FIG. 13, illustrating consecutive current-clamp sweeps at 1-min. intervals using the standard stimulus protocol: the gradual AP plateau run-down shown in the upper panel (“ruptured” configuration) is absent in the lower panel (β-escin-perforated configuration).

This major improvement in externally paced action potential AP shape is stability over a 10-20 min. time interval achieved in β-escin-perforated patch-clamp experiments, because only with a stable shape of the externally paced action potential in control conditions proarrhythmogenic effects of the candidate drug on the shape of the externally paced action potential AP can be rigorously characterized and reverted using dynamic-clamp protocols.

After application of the pharmacological compound and distortion of externally paced action potential AP shape by inhibitory effects on one or multiple ion currents, the sequence of three voltage-clamp protocols is reapplied to the patch-clamped one or several human induced pluripotent stem cell-derived cardiomyocytes hiPSC-CM and the inhibitory effects on the five ion currents measured this way can be approximately estimated.

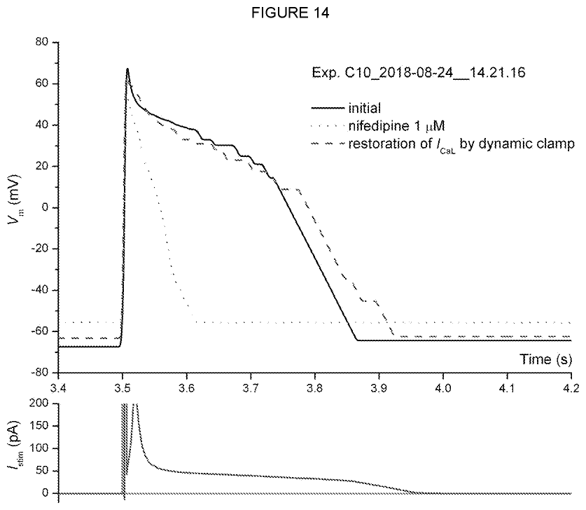

Restoration of the initial externally paced action potential AP shape, as shown in the example illustrated in FIG. 14 may represent a validation criterion for the detection of the proarrhythmogenic risk of said drug candidate. The current restauration stimulus contained is shown in the lower panel of FIG. 14.

### EXAMPLES

The inventors used Cor.4U® cardiomyocytes, a commercial human induced pluripotent stem cell-derived cardiomyocytes hiPSC-CM preparation provided by Ncardia, cultured in multi-well plates precoated with Geltrex® and detached by enzyme treatment with accutase and collagenase II according to manufacturer's instructions.

The experimental phases of the method were performed using a CytoPatch™2 third-generation automated patch-clamp platform based on Cytocentering® technology, using plasma-cleaned standard 2-channel (2K) microfluidic chips with quartz pipette tips of 2.5 μm inner diameter embedded in standard polydimethylsiloxane covers.

The external and internal solutions were physiological and had the following composition (in mM): NaCl 140, KCl 2.5, CaCl₂ 2, MgCl₂ 2, HEPES 10, glucose 10, pH 7.40 at 25° C. titrated with NaOH, osmolality 320 mOsm/kg H₂O adjusted with water/sucrose 1 M (external solution), and K⁺ gluconate 100, KCl 20, CaCl₂ 1, MgCl₂ 1, EGTA 11, HEPES 10, Na₂ phosphocreatine 3, MgATP 4, pH 7.20 at 25° C. titrated with KOH, osmolality 290 mOsm/kg H₂O (internal solution).

The liquid junction potential for this combination of solutions computed with the liquid junction potential calculator application implemented in Clampex 10 (Molecular Devices) (menu: Tools>Junction Potential) was V_{LJP}=13.4 mV.

The external solution, as well as deionized water used by the CytoPatch™2 equipment were degassed overnight by magnetic stirring in flasks connected to a vacuum pump.

The internal solution was kept at −20° C., thawed prior to beginning of experiments and supplemented with β-escin from an aqueous stock solution to 35 μM final concentration.

A complex electrophysiology and pharmacology assay combining multiple voltage-clamp, current-clamp and dynamic-clamp protocols predefined in the CytoLogic software controlling the CytoPatch™2 platform was used.

After chip filling and priming, cell catch and gigaseal formation, the standard voltage-clamp protocol for I_{Na} was repeatedly applied to monitor perforation by plotting the timeline of peak I_{Na} amplitude.

When an adequate perforation was achieved, characterized by an access resistance below 20 MΩ, the sequence of voltage-clamp and current-clamp protocols was initiated.

The parameters of the equivalent electrical circuit (Cₘ, Rₐ) as well as the voltage offset (V_{off}) were directly read as displayed by the CytoLogic software.

The voltage-clamp recordings performed with the 3 standard voltage protocols illustrated in FIGS. 2-4, as well as the current-clamp recording using the standard current stimulus protocol (3 current stimuli of 0.5 ms 2 nA amplitude at 3-s intervals, first stimulus at 500 ms) were exported from the standard ccm format of the CytoLogic software to Axon Text File (ATF) format.

FIG. 2 depicts the voltage-clamp protocol for measuring peak voltage-dependent Na⁺ current amplitude (Nav_standard_v2_MC defined in CytoLogic). The upper panel shows the applied voltage and transmembrane current recorded in a Cor.4U® cardiomyocyte by automated patch-clamp. Peak current is measured as negative peak between 140 and 179 ms, base current as average between 174 and 179 ms, whereas the lower panel shows the voltage dependence of activation and steady-state inactivation of cardiac I_{Na} (human Nav1.5) expressed in CHO cells, based on recordings with specific multi-step voltage protocols, fitted with Boltzmann charge-voltage functions.

FIG. 3 depicts the voltage-clamp protocol for separating L-type Ca²⁺ current (I_{CaL}) from voltage-dependent Na⁺ current (IN) (CaVNaVSeparation_MC defined in CytoLogic). The upper panel shows the applied voltage and transmembrane current recorded in a Cor.4U® cardiomyocyte by automated patch-clamp with an 11-sweep protocol. After an initial depolarizing pulse (200 ms at −20 mV), during the 100-ms step at −40 mV almost all Na⁺ channels remain inactivated (see lower panel of FIG. 2) while most L-type Ca²⁺ channels get removed from inactivation (see lower panel of this figure), therefore a second depolarizing step activates only I_{CaL} as inward current. Peak current is measured as negative peak between 323-343 ms during the 7th sweep (step at +10 mV) and base current as average between 500-519 ms during the same sweep. The lower panel shows the voltage dependence of activation and steady-state inactivation of I_{CaL} measured with specific voltage protocols and solutions in human induced pluripotent stem cell-derived cardiomyocytes (hiPSC-CM), fitted with Boltzmann charge-voltage functions.

FIG. 4 depicts the voltage-clamp protocol for measuring transient outward current (A), delayed rectifier K⁺ current (I_{Kr}+I_{Ks}, mainly I_{Kr}), and “funny” hyperpolarization-activated current (A) (hERG_Standard_m120_m50_MC defined in CytoLogic). The upper panel shows the applied voltage and transmembrane current recorded in a Cor.4U® cardiomyocyte by automated patch-clamp with a 2-sweep protocol. Iₜₒ is measured as positive peak current between 220-500 ms vs. base current measured as average between 10-90 ms during the first sweep, because in the second sweep the outward transient current at +40 mV includes an I_{f} tail current. I_{Kr}+I_{Ks} is measured as peak current at −50 mV during the second sweep, when delayed rectifier K⁺ channels recover rapidly from inactivation, as positive peak between 2210-3000 ms, vs. base current measured as average between 4000-4199 ms during the same sweep, at the base of exponential deactivation. I_{f} is measured as steady current level during the first sweep at −120 mV as average between 4100-4199 ms, vs. starting value of exponential activation measured as positive peak between 2210-3000 ms during the same sweep. The lower panel shows the voltage dependence of activation and steady-state inactivation of I_{Kr} based on experiments on HEK293T cells stably expressing hERG1 with specific voltage protocols (left) and voltage dependence of A conductance g based on experiments in Cor.4U® cardiomyocytes (right), all plots fitted with Boltzmann charge-voltage functions.

The three voltage-clamp recordings in initial conditions were renamed 1.atf, 2.atf, and 3.atf; 3.atf was supplementary processed by low-pass filtering at a cutting frequency of 70 Hz using the Gaussian software filter implemented in Clampfit 10 (Molecular Devices).

The current-clamp recording was also converted to ATF format and supplementary processed by subtracting the offset voltage (V_{off}) (menu: Analyze>Adjust baseline>Subtract fixed value), low-pass filtering as described above, and resampled at 1 ms intervals (menu: Analyze>Data reduction>Substitute average>Reduction factor: 10), and then saved after renaming it AP.atf.

The inventors used a C++ script for automated analysis of the three voltage-clamp recordings to compute ion current conductance surface densities and scaling factors relative to default values in the modified O'Hara-Rudy2011 model for the ion currents I_{Na}, I_{CaL}, Iₜₒ, I_{Kr}, and I_{f}, entering manually the Cₘ and Rₐ values listed above, read directly from the CytoLogic Measured Data window.

The output of the analysis script was written in the file out.dat and used as input for the parameter optimization algorithm.

The inventors then applied a simplified version of the optimization algorithm, running initially a single numeric integration with fixed time step (5 μs) over a total 9-s time interval, using an identical current stimulus protocol as in the real experiment (3 current stimuli of 0.5 ms 2 nA amplitude at 3-s intervals, first stimulus at 500 ms), and using the five ion conductance surface density scaling factors provided by the automated analysis script, the other scaling factors being reasonably guessed.

The output file generated by the optimization algorithm after this first round (a file named output.txt containing time intervals, voltage from experiment—file AP.atf and generated by the model, as well as values of different ion currents, calcium fluxes, ion concentrations, etc.) was imported in Excel, and the experimental and model-generated transmembrane potential over 9 s were plotted in parallel.

Visual inspection of these time plots was used to adjust some parameters, knowing in advance the effects of each parameter on different features of AP and resting potential.

The model with manually readjusted parameters was run once more, and the newly generated file output.txt was reopened in Excel to assess the results.

During the 10-min. time interval required to perform this automated analysis and parameter optimization, the Cor.4U® cardiomyocyte was challenged with nifedipine 1 μM, with repeated current-clamp recordings at 1-min. intervals to monitor the effect of nifedipine on AP shape.

The file output.txt in its final version generated by the optimized model was used with another automated C++ script to generate a current stimulus file (stimfile.csv) in a standard format readable by the CytoLogic software.

This restauration stimulus file contains the sum of ion currents inhibited by the candidate drug (based on percentages of inhibition for each current component defined directly in the script—in this case 100% for I_{CaL} only) with reversed sign and completed by the three standard current stimuli (0.5 ms 2 nA amplitude at 3-s intervals, first stimulus at 500 ms).

The stimulus file was loaded in CytoLogic and applied to the patch-clamped cardiomyocyte in a dynamic-clamp protocol, in order to restore the initial externally paced action potential (AP) shape in the one or several human induced pluripotent stem cell-derived cardiomyocytes hiPSC-CM subjected to the action of the drug candidate.

### Results of the Examples

The parameters of the equivalent electrical circuit in one experiment (C10_2018-08-24_14.21.16_Ch1) were as follows: Cₘ=65.62 pF, Rₐ=12.05 MΩ, V_{off}=44.18 mV.

The computation of each ion current conductance surface density and scaling factor by the C++ automated analysis script occurred as follows.

For I_{Na}: voltage-clamp recording using standard CytoLogic protocol Nav_standard_v2_MC (file 1.aft) was analyzed as shown in FIG. 2:
peak Na⁺ current was measured as negative peak current over the time interval between 140-179 ms: peak_current_early=−6699.82 pA
base current was measured at the base of exponential inactivation during the −10 mV step as average over the time interval 174-179 ms: late_current=−30.52 pA
peak_current_corr=peak_current_early−late_current=−6669.3 pA
corrected voltage of depolarizing step: Vₘ (peak)=−10−(−6.69982 nA×12.05 MΩ) −13.4 mV=57.33 mV
corrected holding voltage: Vₘ (base)=−90−(−0.03052 nA×12.05 MΩ)−13.4 mV=−103.03 mV
activation and steady-state (removal from) inactivation probabilities computed with Boltzmann charge-voltage functions shown in lower panel of FIG. 2:

Pₐ=1/(1+exp(−(Vₘ(peak)+38.5)/6.01998)=1

Pₛₛᵢ=1/(1+exp((Vₘ (base)+75.4)/5.1242)=0.9956
reversal potential for Na⁺ in given conditions: E_{Na}=RT/F ln ([Na⁺]ₒ/[Na⁺]ᵢ)=81.85 mV
driving force: DF=Vₘ (peak)−E_{Na}=57.33 mV−81.85 mV=−24.52 mV
conductance surface density: g_{Na}=peak_current_early_corr (pA)/DF/Cₘ/P_{A}/Pₛₛᵢ=4.163 nS/pF (equivalent to mS/μF)
scaling factor (relative to default value in O'Hara-Rudy2011 model for M type: g_{Na}=75 mS/μF): scgna=0.0555
   For I_{CaL}: voltage-clamp recording using standard CytoLogic protocol CaVNaVSeparation_MC (file 2.atf) (7^{th} sweep at +10 mV) was analyzed as shown in FIG. 3:
peak Ca²⁺ current was measured as negative peak current over the time interval between 323-343 ms: peak_current=−113.73 pA
base current was measured at the base of exponential inactivation during the +10 mV step as average over the time interval 500-519 ms: base_current=9.64 pA
peak_current_corr=peak_current−base_current=−104.09 pA
   corrected voltage of depolarizing step: Vₘ (peak)=10−(−0.11373 nA×12.05 MΩ) −13.4 mV=−2.03 mV
   corrected holding voltage: Vₘ (base)=−40-13.4 mV=−53.4 mV
   activation and steady-state (removal from) inactivation probabilities computed with Boltzmann charge-voltage functions shown in lower panel of FIG. 3:

Pₐ=1/(1+exp(−(Vₘ (peak)+15.2)/5.2)=0.9264

Pₛₛᵢ=1/(1+exp((Vₘ (base)+31.1)/7.6)=0.9495
corrected peak I_{CaL} surface density:
   ilcapeak=peak_current_corr (pA)/Cₘ/P_{A}/Pₛₛᵢ=−1.803 pA/pF
scaling factor for I_{CaL} (by dividing to peak I_{CaL} surface density value for an activating step to −4.418 mV in the O'Hara-Rudy2011 model for mid−myocardial cardiomyocytes ilcapeak=−7.07063 pA/pF): scgcal=0.255
   For Iₜₒ: voltage-clamp recording using standard CytoLogic protocol hERG_Standard_m120_m50_MC (file 3.atf) (first sweep) was analyzed as shown in FIG. 4:
peak Iₜₒ was measured as positive peak current over the time interval between 220-500 ms: Ito_peak=0 pA (no peak Iₜₒ was detected in this experiment)
base current was measured as average over the time interval 10-90 ms during the 100-ms step at −70 mV: Ito_base=−26.99 pA
Ito_peak_corr=Ito_peak−Ito_base (in this case no Ito_peak was detected, therefore Ito_peak_corr=0 pA)
   corrected voltage of depolarizing step: Vₘ (peak)=40−(0 nA×12.05 MΩ)−13.4 mV=26.6 mV
   corrected holding voltage: Vₘ (base)=−70−13.4 mV=−83.4 mV
activation and steady-state (removal from) inactivation probabilities computed with Boltzmann charge-voltage functions from (34):

Pₐ=1/(1+exp(−(Vₘ (peak)+3)/15)=0.88

Pₛₛᵢ=1/(1+exp((Vₘ (base)+33.5)/10)=0.99
reversal potential for K⁺ in given conditions: E_{K}=RT/F ln ([K⁺]ₒ/[K⁺]ᵢ)=−99.49 mV
driving force: DF=Vₘ (peak)−E_{K}=26.6 mV−(−99.49 mV)=126.09 mV
conductance surface density: gₜₒ=Ito_peak_corr (pA)/DF/Cₘ/Pₐ/Pₛₛᵢ=0 nS/pF
scaling factor (relative to default value in O'Hara-Rudy2011 model for M type: gₜₒ=0.08 mS/μF): scgtof=0
   For I_{Kr}: voltage-clamp recording using standard CytoLogic protocol hERG_Standard_m120_m50_MC (file 3.atf) (second sweep) was analyzed as shown in FIG. 4:
peak I_{Kr} was measured as positive peak current over the time interval between 2210-3000 ms (assuming the delayed rectifier peak current at the beginning of the voltage step to −50 mV is contributed entirely by I_{Kr}): IKrpeak=15.78 pA
base current was measured as average over the time interval 4000-4199 ms at the base of the exponential deactivation during the 2-s step at −50 mV: IKrbase=2.29 pA
IKrcorr=IKrpeak−IKrbase=13.49 pA
corrected voltage of repolarizing step: Vₘ (peak)=−50−(0.01349 nA×12.05 MΩ)−13.4 mV=−63.6 mV
   corrected holding voltage: Vₘ (base)=40-13.4 mV=26.6 mV
activation and steady-state (removal from) inactivation probabilities computed with Boltzmann charge-voltage functions shown in lower panel of FIG. 3:

Pₐ=1/(1+exp(−(Vₘ (base)+40.3)/9.18)=0.999

Pₛₛᵢ=1/(1+exp((Vₘ (peak)+25.2)/23.8)=0.83
driving force: DF=Vₘ−E_{K}=−63.5 mV−(−99.49 mV)=35.99 mV
conductance surface density: g_{Kr}=IKrcorr (pA)/DF/Cₘ/P_{A}/Pₛₛᵢ; =0.00689 nS/pF
scaling factor (relative to default value in O'Hara-Rudy2011 model for M type: g_{Kr}=0.0368 mS/μF): scgkr=0.187
   For I_{f}: voltage-clamp recording using standard CytoLogic protocol hERG_Standard_m120_m50_MC (file 3.atf) (first sweep) was analyzed as shown in FIG. 4:
steady I_{f} was measured as average current over the time interval between 4100-4199 ms: Ifstdy=−118.93 pA
the starting point of the exponential I_{f} activation Ifstart was considered the positive peak value over the time interval between 2210-3000 ms: Ifstart=−56.10 pA
Ifcorr=Ifstdy−Ifstart=−62.83 pA
corrected voltage of the hyperpolarizing step:
   Vₘ (steady)=−120−13.4 mV=−133.4 mV
steady-state I_{f} activation probability Pₐ at the corrected voltage step was computed using a Boltzmann charge-voltage function similar to that obtained by fitting the A conductance-voltage plot shown in lower panel of FIG. 3:

Pₐ=1/(1+exp((Vₘ(steady)+96.2)/10.3)=0.97
reversal potential of A computed with a Goldmann-Hodgkin-Katz voltage equation: Eₘ=RT/F ln((P_{Na}[Na⁺]ₒ+P_{K}[K⁺]ₒ)/(P_{Na}[Na⁺]ᵢ+P_{K}[K⁺]ᵢ))=−7.44 mV
   taking permeabilities proportional to I_{f} relative conductances g_{h,Na}=0.3833gₕ and g_{h,K}=0.6167gₕ from (31)
driving force:
   DF=Vₘ (steady)−Eₘ=−133.4 mV−(−7.44 mV)=−125.96 mV
conductance surface density: g_{f}=Ifcorr (pA)/DF/Cₘ/P_{A}=0.00784 nS/pF
scaling factor (relative to default value implemented in modified O'Hara-Rudy2011 model: g_{f}=0.06 mS/μF): scgf=0.131

The scaling factors for these five ion current components computed automatically by the analysis algorithm were extracted from the output file out.dat and inserted in the C++ script of the modified O'Hara-Rudy2011 model used for parameter optimization; the other scaling factors were set to convenient guessed values.

For parameter optimization a rapid two-step approach was used: in the first round estimated parameters were used, yielded by the automated analysis script, and in the second round some parameters were manually adjusted after inspecting the plots of experimentally recorded and model-generated transmembrane potential over 9s applying the same current stimulus protocol.

The output.txt file generated in the last optimization round was used with another automated C++ script to compute the stimulus file stimfile.csv, including 100% of the I_{CaL} component with reversed sign completed with the three brief current stimuli (0.5 ms 2 nA) present in the standard current-clamp protocol.

The current restauration stimulus contained in stimfile.csv is shown in the lower panel of FIG. 14.

The initial shape of the AP is shown in black continuous line in FIG. 14.

While the optimization was performed the Cor.4U® cardiomyocyte was challenged with nifedipine 1 μM, which produced complete block of I_{CaL} and disappearance of the AP plateau (dotted trace in FIG. 14)

By applying the stimulus current waveform from the stimulus file stimfile.csv in a dynamic-clamp protocol restoration of initial externally paced action potential AP shape was obtained (dashed line in FIG. 14).

### REFERENCES

1. S. Polak, M. K. Pugsley, N. Stockbridge, C. Gamett, and B. Wiśniowska, AAPS J, 2015, 17, 1025-32.
2. B. Amuzescu, O. Scheel, and T. Knott, J Phys Chem Biophys., 2014, 4, 1-4.
3. D. Noble, J Pharmacol Sci., 2008, 107, 107-17.
4. J. Kramer, C. A. Obejero-Paz, G. Myatt, Y. A. Kuryshev, A. Bruening-Wright, J. S.

Verducci, and A. M. Brown, Sci Rep, 2013, 3.
5. P. T. Sager, G. Gintant, J. R. Turner, S. Pettit, and N. Stockbridge, Am Heart J, 2014, 167, 292-300.
6. T. O'Hara, L. Virág, A. Varró, and Y. Rudy, PLoS Comput Biol, 2011, 7, 26.
7. R. Wallis, C. Benson, B. Darpo, G. Gintant, Y. Kanda, K. Prasad, D. G. Strauss, and J. P. Valentin, J Pharmacol Toxicol Methods, 2018, 8719, 30626-9.
8. H. Huang, M. K. Pugsley, B. Fermini, M. J. Curtis, J. Koerner, M. Accardi, and S. Authier, J Pharmacol Toxicol Methods, 2017, 87, 11-23.
9. M. J. Windley, N. Abi-Gerges, B. Fermini, J. C. Hancox, J. I. Vandenberg, and A. P. Hill, J Pharmacol Toxicol Methods, 2017, 87, 99-107.
10. Z. Li, S. Dutta, J. Sheng, P. N. Tran, W. Wu, K. Chang, T. Mdluli, D. G. Strauss, and T. Colatsky, Circ Arrhythm Electrophysiol, 2017, 10, 004628.
11. Z. Li, S. Dutta, J. Sheng, P. N. Tran, W. Wu, and T. Colatsky, J Pharmacol Toxicol Methods, 2016, 81, 233-9.
12. S. Dutta, K. C. Chang, K. A. Beattie, J. Sheng, P. N. Tran, W. W. Wu, M. Wu, D. G. Strauss, T. Colatsky, and Z. Li, Front, 2017, 8:616., 10.3389/fphys.2017.00616. eCollection 2017.
13. K. H. Gilchrist, G. F. Lewis, E. A. Gay, K. L. Seligren, and S. Grego, Toxicol Appl Pharmacol, 2015, 288, 249-57.
14. K. Harris, M. Aylott, Y. Cui, J. B. Louttit, N. C. McMahon, and A. Sridhar, Toxicol Sci, 2013, 134, 412-26.
15. A. Mehta, Y. Chung, G. L. Sequiera, P. Wong, R. Liew, and W. Shim, Toxicol Sci, 2013, 131, 458-69.
16. P. Mulder, T. de Korte, E. Dragicevic, U. Kraushaar, R. Printemps, M. L. H. Vlaming, S. R. Braam, and J. P. Valentin, J Pharmacol Toxicol Methods, 2018, 91, 36-42.
17. C. T. Bot, K. Juhasz, F. Haeusermann, L. Polonchuk, M. Traebert, and S. Stoelzle-Feix, J Pharmacol Toxicol Methods, 2018, 8719, 30600-2.
18. H. M. Himmel, J Pharmacol Toxicol Methods, 2013, 68, 97-111.
19. N. Hu, T. Wang, H. Wan, L. Zhuang, R. Kettenhofen, X. Zhang, Y. S. Zhang, W. Xu, M. Gossmann, H. Bohlen, X. Hou, and P. Wang, Biosens Bioelectron, 2018, 117, 354-365.
20. B. Koci, G. Luerman, A. Duenbostell, R. Kettenhofen, H. Bohlen, L. Coyle, B. Knight, W. Ku, W. Volberg, J. R. Woska, Jr., and M. P. Brown, Toxicol Appl Pharmacol, 2017, 329, 121-127.
21. X. Zhang, L. Guo, H. Zeng, S. L. White, M. Fumiss, B. Balasubramanian, E. Lis, A. Lagrutta, F. Sannajust, L. L. Zhao, B. Xi, X. Wang, M. Davis, and Y. A. Abassi, J Pharmacol Toxicol Methods, 2016, 81, 201-16.
22. S. Bedut, C. Seminatore-Nole, V. Lamamy, S. Caignard, J. A. Boutin, O. Nosjean, J. P. Stephan, and F. Coge, Am J Physiol Heart Circ Physiol, 2016, 311, 3.
23. K. Blinova, J. Stohlman, J. Vicente, D. Chan, L. Johannesen, M. P. Hortigon-Vinagre, V. Zamora, G. Smith, W. J. Crumb, L. Pang, B. Lyn-Cook, J. Ross, M. Brock, S. Chvatal, D. Millard, L. Galeotti, N. Stockbridge, and D. G. Strauss, Toxicol Sci, 2017, 155, 234-247.
24. P. W. Burridge, S. Thompson, M. A. Millrod, S. Weinberg, X. Yuan, A. Peters, V. Mahairaki, V. E. Koliatsos, L. Tung, and E. T. Zambidis, PLoS One, 2011, 6, 0018293.
25. M. P. Hortigon-Vinagre, V. Zamora, F. L. Burton, J. Green, G. A. Gintant, and G. L. Smith, Toxicol Sci, 2016, 154, 320-331.
26. A. Lopez-Izquierdo, M. Warren, M. Riedel, S. Cho, S. Lai, R. L. Lux, K. W. Spitzer, I. J. Benjamin, M. Tristani-Firouzi, and C. J. Jou, Am J Physiol Heart Circ Physiol, 2014, 307, 29.
27. E. R. Pfeiffer, R. Vega, P. M. McDonough, J. H. Price, and Rₐ Whittaker, J Pharmacol Toxicol Methods, 2016, 81, 263-73.
28. H. Zeng, M. I. Roman, E. Lis, A. Lagrutta, and F. Sannajust, J Pharmacol Toxicol Methods, 2016, 81, 217-22.
29. W. R. Giles and D. Noble, Biophys J, 2016, 110, 278-80.
30. US2013153067(A1)
31. Y. Kurata, I. Hisatome, S. Imanishi, and T. Shibamoto, Am J Physiol Heart Circ Physiol., 2002, 283, H2074-101. doi: 10.1152/ajpheart.00900.2001
32. O. Scheel, S. Frech, B. Amuzescu, J. Eisfeld, K. H. Lin, and T. Knott, Assay Drug Dev Technol, 2014, 12, 457-69.
33 R. Männikkö, S. Pandey, H. P. Larsson, and F. Elinder, J Gen Physiol., 2005, 125, 305-26. doi: 10.1085/jgp.200409130. Epub 2005 Feb. 14.
34. T. R. Shannon, F. Wang, J. Puglisi, C. Weber, and D. M. Bers, Biophys J., 2004, 87, 3351-71. doi: 10.1529/biophysj.104.047449. Epub 2004 Sep. 3.

1. Method of detecting in vitro the proarrhythmogenic risk of a drug candidate wherein by use of a high-performance automated patch-clamp platform (APCP) able to carry out experimental measurements and recordings of at least five human ventricular ion currents and of an externally paced action potential (AP) on human induced pluripotent stem cell-derived cardiomyocytes (hiPSC-CM) and by use of a computerized algorithm running on a high performance computer unit (HPCU) for rapid parameter optimization of an advanced cardiomyocyte electrophysiology mathematical model to accurately reproduce the shape of said externally paced action potential (AP), the following one-shot sequence of steps is carried out on said human induced pluripotent stem cell-derived cardiomyocytes (hiPSC-CM):
Step 1—Capturing on a microfluidic chip of one or several human induced pluripotent stem cell-derived cardiomyocytes (hiPSC-CM) from a suspension, forming of gigaseals, followed by achieving of whole-cell configuration by b-escin perforation, which allows to stabilize the externally paced action potential (AP) shape over a duration generally between 10 and 20 minutes;
Step 2—Separating, recording, and measuring peak or steady-state amplitudes of the at least five ion currents by applying to said one or several human induced pluripotent stem cell-derived cardiomyocytes (hiPSC-CM) approached in b-escin perforated whole-cell patch-clamp of a series of well-defined voltage-clamp protocols.
Step 3—Computing based on automated analysis of recordings using the series of said voltage-clamp protocols of ion conductance surface densities for each of the at least five said ion currents and of scaling factors for ion conductance surface densities of each of said at least five ion currents relative to default values of said advanced cardiomyocyte electrophysiology mathematical model.
Step 4—Measuring and recording an initial externally paced action potential (AP) of said one or several human induced pluripotent stem cell-derived cardiomyocytes (hiPSC-CM) by applying a standard current-clamp protocol with a single sweep and with predetermined current stimuli.
Step 5
5a—Applying said drug candidate at a given concentration on one or several human induced pluripotent stem cell-derived cardiomyocytes (hiPSC-CM) fulfilling 2 conditions, namely having a resting potential of <−50 mV and a having a ventricular-like initial externally paced action potential (AP) with an Action Potential Duration ratio APD50/APD90>70%, said application of the drug candidate being for a pre-determined period of time, preferably 8 to 15 minutes, and more preferably 10 minutes, and simultaneously, during the same pre-determined period of time:
5b—Measuring and recording the modified externally paced action potential (AP) under the effect of said drug candidate by applying the same current-clamp protocol as for determining initial externally paced action potential (AP), periodically at fixed intervals of time, preferably one-minute, and detecting proarrhythmogenic effects of said candidate drug on the initial externally paced action potential (AP) shape, such as early or delayed afterdepolarizations;
5c—Rapid optimization of parameters of said advanced cardiomyocyte electrophysiology mathematical model using the said scaling factors for ion conductance surface densities of each of said at least five ion currents in order to accurately reproduce the shape of said externally paced initial action potential (AP) on human induced pluripotent stem cell-derived cardiomyocytes (hiPSC-CM) having as result a model-generated shape of said externally paced initial action potential (AP);
Step 6—Measuring and recording amplitudes of the at least five ion currents inhibited after application of said drug candidate using the same series of voltage-clamp protocols as in step 2, comparing them with initial amplitudes in the absence of said drug candidate and calculating percentages of inhibition for each of them;
Step 7—Generating a restauration current stimulus file representing the sum of all ion currents inhibited by the candidate drug with reversed sign and the initial predetermined stimuli, to be used further to restore the initial externally paced action potential (AP) shape in the one or several human induced pluripotent stem cell-derived cardiomyocytes (hiPSC-CM) subjected to the action of the drug candidate by running a simulation with said advanced cardiomyocyte electrophysiology mathematical model with previously optimized parameters and the percentages of inhibition calculated in the previous step or known in advance from previous experiments;
Step 8—Restoring the modified externally paced action potential (AP) to its initial shape, recorded before the application of said drug candidate by applying of the restauration current stimulus file using a dynamic-clamp protocol to the same one human induced pluripotent stem cell-derived cardiomyocyte (hiPSC-CM) on which the drug candidate was applied;
Step 9—Using the calculated percentages of inhibition to compute metrics predictive of proarrhythmogenic risk of said drug candidate such as the net charge integrated over an action potential carried by the sum of several inward and outward ion currents.

2. Method according to claim 1 wherein b-escin perforated whole-cell patch-clamp on the one or several human induced pluripotent stem cell-derived cardiomyocytes (hiPSC-CM) is used in order to stabilize the initial externally paced action potential (AP) shape and the initial peak or steady-state amplitudes of said at least five ion currents over a time interval of 10-20 min. in control conditions, preventing the run-down phenomenon accompanied by progressive shortening of said initial externally paced action potential (AP) shape that occurs in the classical “ruptured” whole-cell patch-clamp approach on said one or several human induced pluripotent stem cell-derived cardiomyocytes (hiPSC-CM).

3. Method according to claim 1 wherein computation of parameters including ion conductance surface densities and scaling factors relative to the advanced cardiomyocyte electrophysiology mathematical model, such as preferably modified O'Hara-Rudy2011 model, of at least five human ventricular ion currents may be achieved in the series of voltage-clamp protocols by using physiological external, respectively extracellular, and internal, respectively pipette solutions, and in the absence of specific blockers, upon measurement of peak or steadystate amplitudes of said at least five ion currents, including at least:
peak rapid voltage-dependent inward Na⁺ current (I_{Na}) with scaling factor of conductance density scgna
peak L-type inward Ca²⁺ current (I_{CaL}) with scaling factor of conductance density scgcal
transient outward K⁺ current (Iₜₒ) with scaling factor of conductance density scgto
“funny” hyperpolarization-activated current (I_{f}) with scaling factor of conductance density scgf
a sum of the rapid (I_{Kr}) and slow (I_{Ks}) components of the delayed rectifier K⁺ current, but where usually I_{Kr} is the dominant component in control conditions, with scaling factor of conductance density scgkr.

4. Method according to claim 1, wherein the optimization of one or several parameters of said advanced cardiomyocyte electrophysiology mathematical model to accurately reproduce the shape of said initial action potential (AP), such as for example scaling factors of conductance surface densities and temperature, is performed in a sequential, parallel, or combined manner on the high-performance computer unit (HPCU), taking as optimality criterion the sum of squared differences between the experimentally recorded initial externally paced action potential (AP) and the model-generated shape of said externally paced initial action potential (AP) and using some or all the previously computed scaling factors.

5. Method according to claim 1, wherein the restauration current stimulus file, representing the sum of all ion currents inhibited by said drug candidate, as generated by said advanced cardiomyocyte electrophysiology mathematical model may be used as a validation criterion for the detection of the proarrhythmogenic risk of said drug candidate.
